(19) United States
(12) Patent Application Publication
(10) Pub. No.: US 2006/0135923 A1
(43) Pub. Date: Jun. 22, 2006

(54) Nonwoven fabrics for use in personal care products

(76) Inventors: Lavada C. Boggs, Marietta, GA (US); Bryan D. Haynes, Advance, NC (US); Hughey K. Jeffries, Marietta, GA (US); Hannong Rhim, Roswell, GA (US); Jacqueline Schmidt Rose, Tampa, FL (US); C. Allen Smith, Cumming, GA (US); Robert D. Wright, Peachtree City, GA (US); Ali Yahiaoui, Roswell, GA (US)

Correspondence Address: KIMBERLY-CLARK WORLDWIDE, INC., 401 NORTH LAKE STREET, NEENAH, WI, 54956, US

(21) Appl. No.: 11/018,768

(22) Filed: Dec. 20, 2004

(51) Int. Cl. A61F 13/15

(52) U.S. Cl. 604/370; 604/365

(57) ABSTRACT

A nonwoven fabric composite that comprises at least one extensible layer of elastomeric fine fibers treated with a wetting agent is provided. The nonwoven fabric composite can be used a barrier layer or a wrap for an absorbent materials to prevent particles form migrating out of the absorbent material.

### FIELD OF THE INVENTION

This invention relates to the field of nonwoven fabrics for use in various products, for example nonwoven fabrics used in personal care products such as diapers.

### BACKGROUND

Attempts to alleviate the loss of superabsorbent particles and the migration of superabsorbent gel have employed various types of barrier materials to shield the superabsorbent material from the wearer's skin. For example, nonwoven fabrics composed of meltblown polypropylene fibers have been used as a “wrap” about an absorbent core to contain superabsorbent particles within the core. The generally hydrophobic nature of the polypropylene however, requires that surfactants be employed to minimize the resistance to the penetration of aqueous liquids there through. The surfactant should exhibit good durability on the nonwoven fabric so that the treated fabric can sustain and remain wettable upon multiple exposures to an aqueous fluid. Otherwise, the surfactant can be washed away after one or two doses of liquid, and subsequent doses of liquid may undesirably be repelled by the fabric. Although meltblown nonwovens can have excellent integrity and particulate retention properties, the nonwovens can be costly to produce.

To provide desired containment of both wet and dry superabsorbent particles, crepe-wadding or tissue has been employed as a lower cost alternative to meltblown nonwovens. Different types of crepe- wadding, such as forming tissue and barrier tissue, have been employed to produce a combination of properties in absorbent articles. Forming tissue is typically a low basis weight, high porosity wadding employed as a substrate onto which a batt of woodpulp fluff fibers are formed in an airlaying process. Designed to allow the passage of a high volume rate of airflow there through, the forming tissue has large numbers of large pores, which provide for a low resistance to airflow but are unable to adequately restrain the movement of relatively smaller superabsorbent particles. As a result, such forming tissues have not provided a sufficient barrier to superabsorbent migration. To address this problem, barrier tissues have been configured with small pores to better contain the superabsorbent particles. The barrier tissues have a low porosity which can be obtained by increasing the tissue basis weight and by modifying the fiber content to create increased fiber coverage. Although the barrier tissue was able to reduce the migration of superabsorbent, its low porosity restricted its versatility and necessitated the use of more complicated manufacturing processes. Additionally, conventional barrier tissues have low strength and tend to easily tear free in areas near the glue line that attaches the barrier tissue to the absorbent core or other portions of a diaper.

Thus, conventional absorbent articles, such as those described above, have required more complicated manufacturing processes and more complex constructions to provide adequate performance. Despite the development of absorbent structures of the types surveyed above, there remains a need for absorbent structures which incorporate improved component layers having a high resistance to the migration of particulate superabsorbent material as well as a high permeability to the passage of air that can be stretched and will recover while maintaining particle resistance and permeability properties.

### SUMMARY OF THE INVENTION

In one embodiment, the present invention provides a personal care product, for example a diaper, that includes: a liquid impervious backing sheet, a nonwoven fabric composite that comprises at least one layer of fine fibers and at least one layer of spunbonded fibers wherein the at least one layer of spunbonded layers is extensible and the at least one layer of fine fibers comprises fibers that comprise a blend of an elastomeric polymer and an internal wetting agent, and an absorbent material disposed between the liquid impervious backing sheet and the nonwoven fabric composite. In certain desirable embodiments, the layer of fine fibers consists essentially of meltblown elastomeric fibers. The nonwoven fabric composite may further include a second layer of spunbonded fibers wherein the second layer of spunbonded fibers is extensible. In this embodiment, it is suggested that the layer of fine fibers consists essentially of a layer of meltblown elastomeric fibers and the nonwoven fabric composite consists essentially of the layer of fine fibers disposed between two spunbonded layers. The layer of fine fibers may consist essentially of a layer of fine fibers selected from the group of metallocene polyethylene fibers and styrenic block copolymer fibers and mixtures thereof. More particularly, the layer of fine fibers may consist essentially of a layer of fine fibers selected from the group styrene/ethylene-propylene/styrene/ethylene-propylene tetrablock copolymer fibers and styrene/ethylene-butylene/styrene/ethylene-butylene tetrablock copolymer fibers and mixtures thereof. These fibers can be surface modified to become water wettable. Fiber surface modification can be achieved via internal melt addition during the extrusion process and suggested internal wetting agents (or surfactants) include, but are not limited to, modified castor oils, hydrogenated ethoxylated castor oils, sorbitan monooleate, ethoxylated siloxanes, and mixtures thereof. Fiber surface modification can also be achieved by topical application of wetting agents (or surfactants) which include, but are not limited to, modified castor oils, hydrogenated ethoxylated castor oils, sorbitan monooleate, alkyl polyglycosides, ethoxylated siloxanes, and mixtures thereof. The nonwoven fabric composite may be necked, creped and/or crimped.

The nonwoven fabric composite may be used as a liquid pervious bodyside liner or a layer between the absorbent material and a liquid pervious bodyside liner. Thus, in certain embodiments, the nonwoven fabric composite is a liquid pervious bodyside liner. In certain other embodiments, the nonwoven fabric composite is a layer between the absorbent material and the liquid pervious bodyside liner. In other embodiments, the nonwoven fabric composite envelops the absorbent material, for example is a wrap for the absorbent. In such embodiments, the nonwoven fabric composite retains any particles such as of superabsorbent particles, synthetic polymer particles, carbon particles and combinations thereof that may be contained in the absorbent core. Desirably, particles of a superabsorbent material in the absorbent material have an average diameter that is greater than the average diameter of the pores in the layer of meltblown layer. The personal care product can be a diaper, a training pant, an absorbent underpant, an adult incontinence products or a feminine hygiene product.

The present invention also provides a nonwoven fabric composite that consists essentially of: an extensible first layer of spunbonded fibers, an extensible second layer of spunbonded fibers, a layer of meltblown fibers disposed between the first layer of spunbonded fibers and the second layer of spunbonded fibers, wherein the layer of meltblown fibers consists essentially of elastomeric fibers include an internal wetting agent or are treated with a wetting agent. In certain embodiments, the layer of fine fibers consists essentially of a layer of fine fibers selected from the group of metallocene polyethylene fibers and styrenic block copolymer fibers. In more particular embodiments, the layer of fine fibers consists essentially of a layer of fine fibers selected from the group of styrene/ethylene-propylene/styrene/ethylene-propylene tetrablock copolymer fibers and styrene/ethylene-butylene/styrene/ethylene-butylene tetrablock copolymer fibers. The first spunbonded layer, the meltblown layer and the second spunbonded layer may be intermittently bonded to form the nonwoven fabric composite. The nonwoven fabric composite, or any layer of the composite, may be densified with a hot air knife. In many of the embodiments, the internal wetting agent is selected from the group consisting of modified castor oils, hydrogenated ethoxylated castor oils, sorbitan monooleate, alkyl polyglycosides and mixtures thereof. In still other embodiments, the nonwoven fabric composite may be necked, creped and/or crimped.

### DEFINITIONS

As used herein the term “bicomponent fibers” refers to fibers which have been formed from at least two polymers extruded from separate extruders but spun together to form one fiber. The polymers are arranged in substantially constantly positioned distinct zones across the cross- section of the bicomponent fibers and extend continuously along the length of the bicomponent fibers. The configuration of such a bicomponent fiber may be, for example, a sheath/core arrangement wherein one polymer is surrounded by another or may be a side by side arrangement or an “islands-in-the-sea” arrangement. Bicomponent fibers are taught in U.S. Pat. No. 5,108,820 to Kaneko et al., U.S. Pat. No. 5,336,552 to Strack et al., U.S. Pat. No. 5,382,400 to Pike et al. and European Patent 0586924. For two component fibers, the polymers may be present in ratios of 75/25, 50/50, 25/75 or any other desired ratios.

As used herein the term “biconstituent fibers” refers to fibers which have been formed from at least two polymers extruded from the same extruder as a blend. The term “blend” is defined below. Biconstituent fibers do not have the various polymer components arranged in relatively constantly positioned distinct zones across the cross-sectional area of the fiber and the various polymers are usually not continuous along the entire length of the fiber, instead usually forming fibrils which start and end at random. Biconstituent fibers are sometimes also referred to as multiconstituent fibers. Fibers of this general type are discussed in, for example, U.S. Pat. No. 5,108,827 to Gessner. Bicomponent and biconstituent fibers are also discussed in the textbook Polymer Blends and Composites by John A. Manson and Leslie H. Sperling, copyright 1976 by Plenum Press, a division of Plenum Publishing Corporation of New York, IBSN 0-306-30831-2, at pages 273 through 277.

As used herein the term “blend” means a mixture of two or more polymers while the term “alloy” means a sub-class of blends wherein the components are immiscible but have been compatibilized. “Miscibility” and “immiscibility” are defined as blends having negative and positive values, respectively, for the free energy of mixing. Further, “compatibilization” is defined as the process of modifying the interfacial properties of an immiscible polymer blend in order to make an alloy.

As used herein, the terms “elastic” and “elastomeric” when referring to a fiber, film or fabric mean a material which upon application of a biasing force, is stretchable to a stretched, biased length which is at least about 150 percent, or one and a half times, its relaxed, unstretched length, and which will recover at least 50 percent of its elongation upon release of the stretching, biasing force.

As used herein, the term “machine direction” or “MD” means the length of a fabric as it is produced. The term “cross machine direction”, “cross-direction” or “CD” means across the width of fabric, i.e. a direction generally perpendicular to the MD.

As used herein the term “meltblown fibers” means fibers formed by extruding a molten thermoplastic material through a plurality of fine, usually circular, die capillaries as molten threads or filaments into a high velocity gas (e.g. air) stream which attenuates the filaments of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin. Meltblown fibers are usually microfibers which are generally smaller than 30 microns in diameter and in many instances smaller than 10 microns in diameter.

As used herein the term “microfibers” means small diameter fibers having an average diameter not greater than about 50 microns, for example, having an average diameter of from about 0.5 microns to about 50 microns, or more particularly, microfibers may have an average diameter of from about 2 microns to about 40 microns. The diameter of, for example, a polypropylene fiber given in microns, may be converted to denier by squaring, and multiplying the result by 0.00629, thus, a 15 micron polypropylene fiber has a denier of about 1.42 (15².times.0.00629=1.415).

As used herein, the terms “necking” or “neck stretching” interchangeably refer to a method of elongating a nonwoven fabric, generally in the machine direction, to reduce its width in a controlled manner to a desired amount. The controlled stretching may take place under cool, room temperature or greater temperatures and is limited to an increase in overall dimension in the direction being stretched up to the elongation required to break the fabric, which in most cases is about 1.2 to 1.4 times. When relaxed, the web retracts toward its original dimensions. Such a process is disclosed, for example, in U.S. Pat. No. 4,443,513 to Meitner and Notheis and another in U.S. Pat. No. 4,965,122 to Morman.

As used herein the term “neck softening” means neck stretching carried out without the addition of heat to the material as it is stretched.

As used herein, the term “neckable material” means any material which can be necked.

As used herein, the term “necked material” refers to any material which has been constricted in at least one dimension by processes such as, for example, drawing or gathering.

As used herein the term “nonwoven fabric or web” means a web having a structure of individual fibers or threads which are interlaid, but not in an identifiable or regularly repeating manner as in a knitted fabric. Nonwoven fabrics or webs have been formed from many processes such as for example, meltblowing processes, spunbonding processes, and bonded carded web processes. The basis weight of nonwoven fabrics is usually expressed in ounces of material per square yard (osy) or grams per square meter (gsm) and the fiber diameters useful are usually expressed in microns. (Note that to convert from osy to gsm, multiply osy by 33.91).

As used herein, the term “personal care product” means diapers, baby bibs, training pants, absorbent underpants, adult incontinence products, wipers and feminine hygiene products.

As used herein the term “polymer” generally includes but is not limited to, homopolymers, copolymers, such as for example, block, graft, random and alternating copolymers, terpolymers, etc. and blends and modifications thereof. Furthermore, unless otherwise specifically limited, the term “polymer” shall include all possible geometrical configurations of the material. These configurations include, but are not limited to isotactic, syndiotactic, atactic and random symmetries.

As used herein the term “recover” refers to a contraction of a stretched material upon termination of a biasing force following stretching of the material by application of the biasing force. For example, if a material having a relaxed, unbiased length of one (1) inch was elongated 50 percent by stretching to a length of one and one half (1.5) inches the material would have been elongated 50 percent and would have a stretched length that is 150 percent of its relaxed length. If this exemplary stretched material contracted, that is recovered to a length of one and one tenth (1.1) inches after release of the biasing and stretching force, the material would have recovered 80 percent (0.4 inch) of its elongation.

As used herein the term “spunbonded fibers” refers to small diameter fibers which are formed by extruding molten thermoplastic material as filaments from a plurality of fine, usually circular capillaries of a spinneret with the diameter of the extruded filaments then being rapidly reduced as by, for example, in U.S. Pat. No. 4,340,563 to Appel et al., and U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartman, U.S. Pat. No. 3,502,538 to Levy, and U.S. Pat. No. 3,542,615 to Dobo et al. Spunbond fibers are generally continuous and larger than 7 microns, more particularly, often between about 10 and 30 microns.

As used herein, the term “stitchbonded” means, for example, the stitching of a material such as in accordance with U.S. Pat. No. 4,891,957 to Strack et al. or U.S. Pat. No. 4,631,933 to Carey, Jr.

As used herein the term “un-necking” means a process applied to a reversibly necked material to extend it to at least its original, pre-necked dimensions by the application of a stretching force in a longitudinal or cross-machine direction which causes it to recover to within at least about 50 percent of its reversibly necked dimensions upon release of the stretching force.

### TEST METHODS

Fluid Intake Flowback Evaluation Test

The Fluid Intake Flowback Evaluation (FIFE) Test determines the amount of time required for an absorbent structure, and more particularly a sample thereof, to take in (but not necessarily absorb) a known amount of test solution (0.9 weight percent solution of sodium chloride in distilled water at room temperature). The FIFE test is known and a suitable apparatus for performing the FIFE Test is shown in FIGS. 5 and 6 of U.S. Publication no. 2004/0214499 A1, generally indicated as 200. The test apparatus 200 comprises upper and lower assemblies, generally indicated at 202 and 204 respectively, wherein the lower assembly comprises a generally 14 inch (35.56 cm) by 8 inch (20.32 cm) rectangular plate 206 constructed of a transparent material such as Plexiglas and a generally 6 inch (15.24 cm) by 3 inch (7.62 cm) rectangular platform 207 centered on the plate for centering the absorbent structure sample during the test.

The upper assembly 202 comprises a generally rectangular plate 208 constructed similar to the lower plate 206 and having a central opening 210 formed therein. A cylinder 212 having an inner diameter of about 2 inches (about 5.08 cm) and a height of about 4 inches (about 10.16 cm) is secured to the upper plate 208 at the central opening 210 and extends upward substantially perpendicular to the upper plate. The central opening 210 of the upper plate 208 should have a diameter at least equal to the inner diameter of the cylinder 212 where the cylinder is mounted on top of the upper plate. However, the diameter of the central opening 210 may instead be sized large enough to receive the outer diameter of the cylinder 212 within the opening so that the cylinder is secured to the upper plate 208 within the central opening.

Pin elements 214 are located near outside corners of the lower plate 206, and corresponding recesses 216 in the upper plate 208 are sized to receive the pin elements to properly align and position the upper assembly 202 on the lower assembly during testing. The weight of the upper assembly 202 (e.g., the upper plate 208 and cylinder 212) is approximately 845 grams.

To run the FIFE Test, an absorbent structure sample 218 (either formed to the desire size or removed from an absorbent article and cut to the desired size) having length and width dimensions of about 14 inches (35.56 cm) by about 3 inches (7.68 cm) is weighed, with the tissue wrap on, and the weight is recorded in grams. The sample 218 is then centered on the platform 207 of the lower assembly. The upper assembly 202 is placed over the sample in opposed relationship with the lower assembly, with the pins 214 of the lower plate seated in the recesses 216 formed in the upper plate 208 and the cylinder 212 generally centered over the sample. Approximately 75 mL of the test solution (referred to herein as a first insult) is poured into the top of the cylinder 212 (e.g., generally at the height of the top of the cylinder) and allowed to flow down into the absorbent structure sample 218. A stopwatch is started when the first drop of solution contacts the sample 218 and is stopped when the liquid ring between the edge of the cylinder 212 and the sample disappears. The reading on the stopwatch is recorded to two decimal places and represents the intake time (in seconds) required for the first insult to be taken into the absorbent structure sample 218.

A time period of twenty minutes is allowed to elapse, after which a second insult equal to the first insult is poured into the top of the cylinder 212 and again the intake time is measured as described above. The procedure is repeated for a third insult and then a fourth insult as well. An intake rate (in milliliters/second) for each of the four insults is determined by dividing the amount of solution (e.g., 75 mL) used for each insult by the intake time measured for the corresponding insult.

At least 3 samples of each absorbent structure are subjected to the FIFE Test and the results are averaged to determine the intake time and intake rate of the absorbent structure.

Pore Size Analysis Via Optical Microscopy

Materials were sampled by cutting out five randomly spaced squares of material. The square pieces of material were approximately 1-2 square inches in size. Samples were prepped for image analysis by gold coating them on a single surface to reduce the level of light transmitted through bond points. A Denton Vacuum Desk II sputter coater was used to apply a 350 angstrom thick gold layer.

All pore size and percent open area measurements were made using a Quantimet 600 Image Analysis (IA) System and the custom-written Quantimet User Interactive Programming System (QUIPS) routines ‘PORES’ and ‘PORES10X.’ The routine ‘PORES10X’ was used exclusively for the LAG SMS sample which required a higher magnification. Copies of the routines are shown in Appendices 1 and 2. The image analysis routines were written to control all aspects of the data collection process (e.g., auto-stage control, image acquisition, image processing, pore detection, pore measurements, and data exportation). A detailed description of the instrumentation and optical set-up used is provided below.
Instrumentation: Quantimet 600 IA System (Leica, Inc. Deerfield, Ill.)
System Software: QWIN Version 1.06
IA Routines: PORES and PORESLOX (LAG SMS only)
Microscope: Olympus BH-2 fitted with a 2× objective lens and a mag changer setting of 1.5 for a total magnification of 3×. For the LAG SMS sample, a 10× objective was used.
Field-of-view (FOV) Size: 3×=7.5 mm²; 10×=0.7 mm²
Lighting: Transmitted light through a low-magnification condenser.
Video Camera: SONY 3CCD, model DXC-930P
Auto-Stage: Mertzhauser auto-stage with a ¼″ glass cover plate.

The gold-coated sample pieces were placed under the microscope on the auto-stage and covered with the glass plate. For each material square analyzed using 3× magnification, 25 adjacent FOV were sampled in a 5×5 matrix on the auto-stage. For the LAG SMS material, 100 FOV were sampled for each square. For statistical purposes, each sample square was treated as a single pore size data point even though hundreds of pores were measured within each square.

Porosity measurement parameters equivalent-circular diameter (ECD) and convoluted pore width (CPW) were defined by the following derived parameters:
ECD=(4×Area/π)^{1/2}
CPW=[0.9×(4×Area/Perimeter)×(4π×Area/Perimeter²)_{1/4}]
ECD is a fairly know parameter to those skilled in the art, although CPW was developed exclusively for the measurement of asymmetrical and convoluted shaped pores.
Mist

The articles and systems of the present invention were examined using a cradle. The cradle that is used for the testing is acrylic and is formed to simulate the curvature of a user such as an infant. Such a cradle is illustrated in FIG. 5 of U.S. patent application Ser. No. 10/026862 filed Dec. 20, 2001. The cradle has a width into the page of the drawing as shown of 33 cm and the ends are blocked off, a height of 19.5 cm, an inner distance between the upper arms of 28 cm and an angle between the upper arms of 60 degrees. The cradle has a 6.5 mm wide slot at the lowest point running the length of the cradle into the page. The material to be tested is placed in the cradle with the topsheet up and the backsheet towards the acrylic cradle. The target or crotch is centered over the slot at the lowest point of the cradle. The ends of the material are placed above the crotch, to simulate the position of the product on a user. A first insult, 60 ml Blood Bank Saline (pH 7.0-7.2, 8.5g/L of A.C.S. grade Sodium Chloride, CAS #7647-14-5 in ultrapure reagent grade water, CAS #7732-18-5), is introduced into the product using a Masterflex Digi-Staltic® 7526-00 pump with Masterflex 6409-17 tubing (both available from Cole-Parmer Instrument Company, Vernon Hills, Ill.) connected to a spray nozzle with a 3 mm orifice. The insult is introduced at a rate of 15 ml/sec. One minute after the insult, the material is removed from the cradle and placed flat on an x-ray unit. An x-ray is taken of the xy-plane of the diaper and is analyzed for fluid distribution. The material is placed back in the cradle in substantially the same position as the original placement. A second insult of 60 ml of saline is introduced into the diaper after 20 minutes in the cradle. One more minute after the second insult is complete, the product is removed and x-rayed a second time and analyzed for fluid distribution. The x-ray system was operated with an exposure time of 5 seconds, with a tube voltage of 30 KV and current of 12 mA. Prior to each x-ray, a metal rod was placed across the entire width or cross-direction of the product at the point of insult such that the insult point was discernable in the x-ray picture.

The x-ray systems used in these Examples are available from Tronix Inc. of 31 Business Park Drive, Branford, Conn. 06045 as Model No. 10561 HF 100 with an appropriate enclosure. This system uses BIO-SCAN OPTIMATE version 6.2 software, available from Optumus, Inc., of Ft. Collins, Colo.

### BRIEF DESCRIPTION OF THE DRAWINGS

The invention will be more fully understood and further advantages will become apparent when reference is made to various embodiments described in the following description and the accompanying drawings in which:

FIG. 1 illustrates a schematic diagram of an exemplary forming machine that can be used to form a laminate of the present invention.

FIG. 2 illustrates a cross-section view of a three-layer embodiment of a laminate of the present invention showing a three-layer configuration including an internal fine fiber barrier layer and two continuous fiber layers.

FIG. 3 illustrates a cross-section view of an alternative two-layer embodiment of a laminate of the present invention including a fine fiber barrier layer and one continuous fiber layer.

FIG. 4 illustrates a partially cut away top plan view of an exemplary personal care absorbent article, in this case a diaper, which utilizes a laminate according to the present invention.

FIG. 5 illustrates a perspective view of another absorbent article according to yet another embodiment of the present invention.

FIG. 5A illustrates a cross-sectional side view of an absorbent article according to the present invention.

FIG. 5B illustrates a cross-sectional side view of another absorbent article according to the present invention.

FIG. 5C illustrates a cross-sectional side view of another absorbent article according to the present invention.

FIG. 6 present the results of the Fluid Intake Flowback Evaluation (FIFE) testing.

FIG. 7 presents the results of modified mist testing.

FIG. 8 illustrates a top plan of a suitable apparatus for conducting a FIFE Test.

FIG. 9 illustrates a section taken in the plane of line 6-6 of FIG. 9.

### DETAILED DESCRIPTION

Composite fabrics of the present invention comprise a construction that includes at least one layer of an elastomeric thermoplastic polymer layer and at least one layer of extensible spunbond nonwoven fabric. In certain desirable embodiments, the layer of extensible spunbonded nonwoven fabric comprises spunbond fibers that are crimped bicomponent fibers or other crimped multicomponent fibers and include mixtures thereof. Desirably, composites of the present invention have elastic properties and desirably stretch and recover in both the machine-direction and cross-direction. More desirably, composites of the present invention can stretch and substantially recover their original dimensions in both the machine- and cross-directions while providing a barrier to superabsorbent particles (SAP), that is resist SAP strikethrough. Even more desirably, composite materials of the present invention exhibit acceptable fluid intake and air permeability properties. Such composites are particularly useful in personal care articles such as diapers and may be used as a bodyside liner, as core wrap, or as a transfer layer in diapers, training pants, swimming pants, absorbent underpants, adult incontinence products, feminine hygiene products and so forth. The fluid intake properties of nonwoven composites fabrics of the present invention can be improved by the inclusion of one or more internal wetting agents in any or all of layers of the composite fabric or by surface treatment of any or all of the layers of the composite or by a combination thereof.

Elastomeric thermoplastic polymers useful in the practice of this invention may be those made from styrenic block copolymers, polyurethanes, polyamides, copolyesters, ethylene vinyl acetates (EVA) and so forth and may be a meltblown web, a spunbond web, a film or a foam layer and may itself be composed of one or more thinner layers of elastomeric thermoplastic polymer. Generally, any suitable elastomeric fiber, film or foam forming resins or blends containing the same may be utilized to form the nonwoven webs of elastomeric fibers, elastomeric film or elastomeric foam. Suggested styrenic block copolymers include styrene/butadiene/styrene (SBS) block copolymers, styrene/isoprene/styrene (SIS) block copolymers, styrene/ethylene-propylene/styrene (SEPS) block copolymers, styrene/ethylene-butadiene/styrene (SEBS) block copolymers. For example, useful elastomeric fiber forming resins include block copolymers having the general formula A-B-A′ or A-B, where A and A′ are each a thermoplastic polymer endblock which contains a styrenic moiety such as a poly (vinyl arene) and where B is an elastomeric polymer midblock such as a conjugated diene or a lower alkene polymer. Block copolymers of the A-B-A′ type can have different or the same thermoplastic block polymers for the A and A′ blocks, and the present block copolymers are intended to embrace linear, branched and radial block copolymers. In this regard, the radial block copolymers may be designated (A-B)ₘ-X, wherein X is a polyfunctional atom or molecule and in which each (A-B)ₘ- radiates from X in a way that A is an endblock. In the radial block copolymer, X may be an organic or inorganic polyfunctional atom or molecule and m is an integer having the same value as the functional group originally present in X. It is usually at least 3, and is frequently 4 or 5, but not limited thereto. Thus, in the present invention, the expression “block copolymer” and particularly “A-B-A′” and “A-B” block copolymer, is intended to embrace all block copolymers having such rubbery blocks and thermoplastic blocks as discussed above, which can be extruded (e.g., by meltblowing), and without limitation as to the number of blocks. U.S. Pat. No. 4,663,220 to Wisneski et al. discloses a web including microfibers comprising at least about 10 weight percent of an A-B-A′ block copolymer where “A” and “A′” are each a thermoplastic endblock which comprises a styrenic moiety and where “B” is an elastomeric poly(ethylene-butylene) midblock, and from greater than 0 weight percent up to about 90 weight percent of a polyolefin which when blended with the A-B-A′ block copolymer and subjected to an effective combination of elevated temperature and elevated pressure conditions, is adapted to be extruded, in blended form with the A-B-A′ block copolymer. Polyolefins useful in Wisneski et al. may be polyethylene, polypropylene, polybutene, ethylene copolymers, propylene copolymers, butene copolymers, and mixtures thereof.

Commercial examples of such elastomeric copolymers are, for example, those known as KRATON® materials which are available from Kraton Polymers of Houston, Tex. KRATON® block copolymers are available in several different formulations, a number of which are identified in U.S. Pat. No. 4,663,220, hereby incorporated by reference. A particularly suitable elastomeric layer may be formed from, for example, elastomeric poly(styrene/ethylenebutylene/styrene) block copolymer available from the Kraton Polymers of Houston, Tex. under the trade designation KRATON® 2760. Other exemplary elastomeric materials which may be used to form an elastomeric layer include polyurethane elastomeric materials such as, for example, those available under the trademark ESTANE from B. F. Goodrich & Co., polyamide elastomeric materials such as, for example, those available under the trademark PEBAX from the Rilsan Company, and polyester elastomeric materials such as, for example, those available under the trade designation HYTREL from E. I. DuPont De Nemours & Company. Formation of an elastomeric nonwoven web from polyester elastomeric materials is disclosed in, for example, U.S. Pat. No. 4,741,949 to Morman et al., hereby incorporated by reference.

Elastomeric layers may also be formed from elastomeric copolymers of ethylene and at least one vinyl monomer such as, for example, vinyl acetates, unsaturated aliphatic monocarboxylic acids, and esters of such monocarboxylic acids. The elastomeric copolymers and formation of elastomeric nonwoven webs from those elastomeric copolymers are disclosed in, for example, U.S. Pat. No. 4,803,117. Particularly useful elastomeric meltblown thermoplastic webs are composed of fibers of a material such as disclosed in U.S. Pat. No. 4,707,398 to Boggs, U.S. Pat. No. 4,741,949 to Morman et al., and U.S. Pat. No. 4,663,220 to Wisneski et al. In addition, the elastomeric meltblown thermoplastic polymer layer may itself be composed of one or more thinner layers of elastomeric meltblown thermoplastic polymer which have been sequentially deposited one atop the other or laminated together by methods known to those skilled in the art.

The thermoplastic copolyester elastomers include copolyetheresters having the general formula: 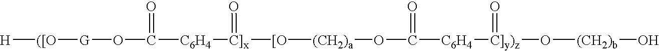
where “G” is selected from the group consisting of poly(oxyethylene)-alpha,omega-diol, poly(oxypropylene)-alpha, omega-diol, poly(oxytetramethylene)-alpha, omega-diol and “a” and “b” are positive integers including 2, 4 and 6, “x”, “y”, and “z” are positive integers including 1-20. Such materials generally have an elongation at break of from about 600 percent to 750 percent when measured in accordance with ASTM D-638 and a melt point of from about 350° F. to about 400° F. (176° C. to 205° C.) when measured in accordance with ASTM D-2117. Commercial examples of such copolyester materials are, for example, those known as ARNITEL formerly available from Akzo Plastics of Arnhem, Holland and now available from DSM of Sittard, Holland, or those known as HYTREL which are available from E.I. Dupont de Nemours of Wilmington, Del. Examples of suitable foams include those produced by the General Foam Corporation of Paramus, N.J. Such foams are polyurethane foams under the trade designation “4000 Series”. Such foams are described in U.S. Pat. No. 4,761,324 to Rautenberg et al. at column 6, lines 53-68, hereby incorporated by reference.

An elastomeric meltblown layer may be stitchbonded in accordance with U.S. Pat. No. 4,891,957 to Strack et al. Stitchbonding imparts strength and durability to the stitchbonded product and stitchbonding in the present invention is believed to impart increased abrasion resistance to the composite or composite nonwoven fabric. While stitchbonding generally is used to join two or more materials together, in this embodiment of the present invention the elastomeric meltblown layer is stitchbonded alone and then used in the fabrication of the composite or composite nonwoven fabric.

The extensible spunbond nonwoven layer may be produced by methods known in the art and described in a number of the references cited above. One particularly desirable method of producing an extensible spunbonded fabric includes spunbonding bicomponent fibers that are crimpable. An exemplary method of spunbonding bicomponent fibers and crimping the bicomponent fibers is disclosed in U.S. Pat. No. 5,382,400. Briefly, the spunbond process generally uses a hopper which supplies polymer to a heated extruder. The extruder supplies melted polymer to a spinneret where the polymer is fiberized as it passes through fine openings usually arranged in one or more rows in the spinneret, forming a curtain of filaments. The filaments are usually quenched with air at a low pressure, drawn, usually pneumatically, and deposited on a moving foraminous mat, belt or “forming wire” to form the nonwoven fabric. The fibers produced in the spunbond process are usually in the range of from about 10 to about 30 microns in diameter, depending on process conditions and the desired end use for the fabrics to be produced from such fibers. For example, increasing the polymer molecular weight or decreasing the processing temperature result in larger diameter fibers. Changes in the quench fluid temperature and pneumatic draw pressure can also affect fiber diameter. Polymers useful in the spunbond process generally have a process melt temperature of between about 350° F. to about 610° F. (175° C. to 320° C.) and a melt flow rate, as defined above, in the range of about 10 to about 150, more particularly between about 10 and 50. Examples of suitable polymers include polypropylenes, polyethylenes and polyamides.

Bicomponent fibers and other multicomponent fibers are desirable in the practice of this invention. Bicomponent fibers are commonly polypropylene and polyethylene arranged in a sheath/core, “islands in the sea” or side by side configuration. Suitable commercially available materials include polypropylene designated PP-3445 from the Exxon Chemical Company of Baytown, Tex., ASPUN 6811A and 2553 linear low density polyethylene from the Dow Chemical Company of Midland, Mich., 25355 and 12350 high density polyethylene from the Dow Chemical Company, DURAFLEX DP 8510 polybutylene available from the Shell Chemical Company of Houston, Tex., and ENATHENE 720-009 ethylene n-butyl acrylate from the Quantum Chemical Corporation of Cincinnati, Ohio.

Certain biconstituent fibers may also be used in the practice of this invention. Blends of a polypropylene copolymer and polybutylene copolymer in a 90/10 mixture have been found effective. Any other blend would be effective as well provided they may be spun and provide crimped or crimpable fibers. In certain embodiments, the fibers of the spunbond layer used in the practice of this invention are crimped because crimped fiber webs, when laminated to an elastomeric meltblown layer, have enough “give” to stretch to a larger dimension without breaking.

The crimping of a spunbond fiber may be accomplished through a number of methods. One method is to produce a spunbond web onto a forming wire and then pass the web between two drums or rollers with differing surfaces. The rollers bend the fibers of the web as it passes therebetween and produces the desired crimp. Another method of creating fiber crimp is to mechanically stretch each fiber. When bicomponent spunbond fibers are used in the practice of this invention, crimping may be accomplished by heating the fibers. The two polymers making up the bicomponent fibers may be selected to have different coefficients of expansion and so upon heating create crimps in the fibers. This heating may be done after the formation of the web on the forming wire at a temperature of from about 110° F. (43° C.) up to a temperature less than the melting point of the lower melting component of the fibers. This heating may alternatively be done as the fibers drop from the spinneret to the forming wire as taught in U.S. Pat. No. 5,832,400 to Pike et al. In the Pike process, heated air in the range of from about 110° F. (43° C.) up to a temperature less than the melting point of the lower melting component of the fibers is directed at the fibers as they fall, causing the two polymers to expand differentially to one another and the fiber to crimp.

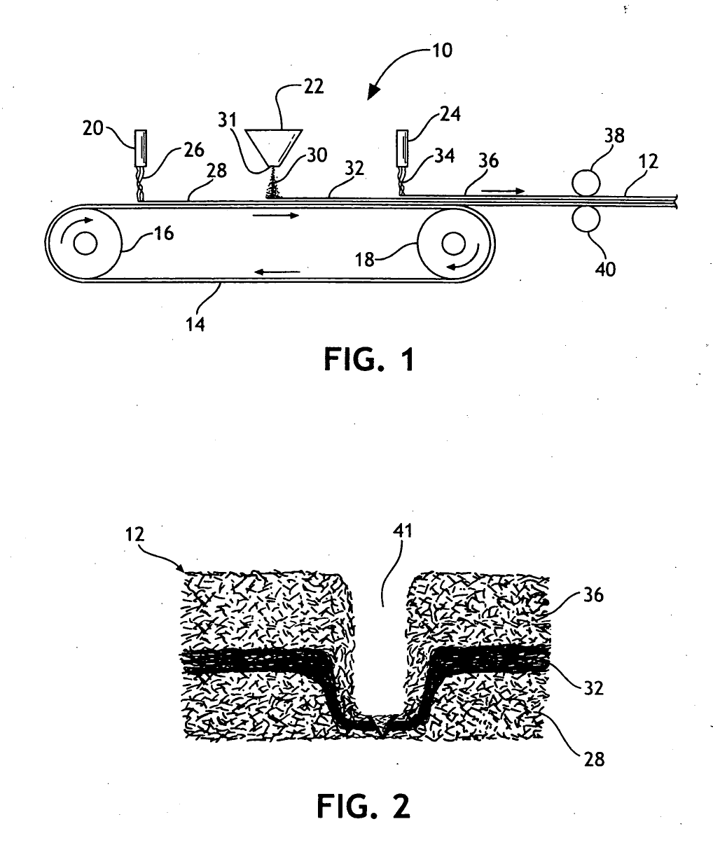

Exemplary composite nonwoven fabrics in accordance with the present invention may be made by first depositing onto a forming wire a layer of crimped spunbond fibers. A layer of elastomeric meltblown fibers is deposited on top of the crimped spunbond fibers. Lastly, another layer of crimped spunbond fibers is deposited atop the meltblown layer and this layer is usually preformed. There may be more than one layer of elastomeric meltblown fibers. None of the layers of the composite nonwoven fabric need to be stretched in any direction during the production process of the fabric, including the bonding step, to provide the composite nonwoven fabric with elasticity. Alternatively, any or all of the layers of the composite fabric may be produced independently and brought together in a separate lamination step. Turning to FIG. 1, there is shown schematically a forming machine 10 which may be used to produce a nonwoven fabric laminate 12 having a fine fiber meltblown layer 32 and outer continuous filaments spunbond layer 28 in accordance with the present invention. Particularly, the forming machine 10 includes of an endless foraminous forming belt 14 wrapped around rollers 16 and 18 so that the belt 14 is driven in the direction shown by the arrows. The illustrated forming machine 10 has three stations: spunbond station 20, meltblown station 22, and spunbond station 24 to produce a spunbond/meltblown/spunbond (SMS) laminate as described in U.S. Pat. No. 4,041,203 which is hereby incorporated by reference herein. It should be understood that more than three forming stations may be utilized to build up additional layers to produce a laminate of more layers or having a higher basis weight and each forming station can have multiple banks or dies. Alternatively, each of the laminate layers may be formed separately, rolled, and later converted to the fabric laminate off-line. In addition the fabric laminate 12 could be formed of more than or less than three layers depending on the requirements for the particular end use for the fabric laminate 12. For example, for some applications it may be preferred to have a laminate of one fine fiber meltblown web layer and one spunbond continuous filament web layer. Particularly, for extremely lightweight applications and/or high liquid permeability applications a two-layer laminate is desirable.

The spunbond stations 20 and 24 can be conventional extruders with spinnerets which form continuous filaments of a polymer and deposit those filaments onto the forming belt 14 in a random interlaced fashion. The spunbond stations 20 and 24 may include one or more spinnerets heads depending on the speed of the process and the particular polymer or polymers being used. Forming spunbonded material is conventional in the art, and the design of such a spunbonded forming station is within the ability of those of ordinary skill in the art. The nonwoven spunbonded webs 28 and 36 can be formed using known methods such as those described and illustrated in the above-mentioned patents.

Although the fabric of this invention does not need to be neck-stretched, neck softened or un-necked to provide the desired stretch properties, in certain embodiments it would be desirable to neck stretch, crepe or otherwise mechanically soften the composite fabric of the present invention in order to provide a softer nonwoven fabric. The requirement of the fabric being unstretched during fabrication into a composite means that the fabric is not subjected to any additional or excessive stretching force beyond that normally provided by the type of mechanism that is usually used to produce the composite nonwoven fabric, i.e. rollers and winders which move the fabric along the path of the process from pre- to post-lamination. Methods of necking are known. An exemplary method of reversibly necking a spunbonded nonwoven fabric is described in U.S. Pat. No. 4,965,122 to Morman et al. and an exemplary method of forming a composite elastic neck-bonded material is described in U.S. Pat. No. 5,226,992 to Morman et al. Methods of creping are also known. An exemplary method of creping a spunbonded nonwoven fabric to improve the softness of the spunbonded nonwoven is described in U.S. Pat. No. 6,592,697.

After the addition of the last layer of crimped spunbond fibers, the layers are bonded to produce the composite nonwoven fabric. The bonding may be done thermally such as by through-air bonding or by point bonding using patterned calender rolls. Through-air bonding or “TAB” is discussed in U.S. Pat. No. 5,382,400 to Pike et al. and is a process of bonding a nonwoven bicomponent fiber web which is wound at least partially around a perforated roller which is enclosed in a hood. Air which is sufficiently hot to melt one of the polymers of which the fibers of the web are made is forced from the hood, through the web and into the perforated roller. The air velocity is between 100 and 500 feet per minute and the dwell time may be as long as 6 seconds. The melting and resolidification of the polymer provides the bonding. Since TAB requires the melting of at least one component to accomplish bonding, it is desirable that one or more of the layers of the composite fabric include bicomponent fibers.

Thermal point bonding using calender rolls with various patterns have been developed. One example is the expanded Hansen Pennings pattern with about a 15 percent bond area with about 100 bonds/square inch as taught in U.S. Pat. No. 3,855,046 to Hansen and Pennings. Another common pattern is a diamond pattern with repeating and slightly offset diamonds. The bonding of the composite nonwoven fabric may alternatively be done ultrasonically, by print adhesive bonding by any other method known in the art to be effective except the method of hydroentanglement.

Fabric of the present invention may be treated, either the individual layers prior to lamination or the entire fabric after lamination, with various chemicals in accordance with known techniques to give properties for specialized uses. Such treatments include water repellant chemicals, softening chemicals, fire retardant chemicals, oil repellant chemicals, antistatic agents and mixtures thereof. Pigments may also be added to the fabric as a post-bonding treatment or alternatively added to the polymer of the desired layer prior to fiberization. In one desirable embodiment, the meltblown layer includes one or more internal and/or topical wetting agents or is surface treated (or modified) to improve wettability of the meltblown layer and of the overall composite nonwoven fabric. The wetting agent may include, but is not limited to, modified castor oils, hydrogenated ethoxylated castor oils, sorbitan monooleate, alkyl polyglycosides, ethoxylated siloxanes and so forth including mixtures of wetting agents. Suggested commercially available wetting agents include, but are not limited to, AHCOVEL Base N-62 (also referred to as “Ahcovel”) and MASIL SF-19 (also referred to as “SF 19”) and Dow Corning 193 Surfactant that can be obtained from Dow Corning, Midland, Mich. Other suggested agents that can be used to improve the wettability of the composite or any of the layers of the composite include, but are not limited to, the siloxanes described in U.S. Pat. No. 5,336,707 to Nohr et al. which may be included in the melted thermoplastic compositions use to make any portion of the layers of the composite.

In another embodiment, a laminate of the present invention is surface treated with one or more surfactants to improve the wettability of the laminate. One suggested surfactant that can be used to surface treat a nonwoven of the present invention is a surfactant mixture that contains a mixture of both AHCOVEL Base N-62 and GLUCOPON 220 UP surfactant in a 3:1 ratio based on a total weight of the surfactant mixture. AHCOVEL Base N-62 can be obtained from Uniqema Inc., a business having offices in New Castle, Del., and includes a blend of hydrogenated ethoxylated castor oil and sorbitan monooleate. GLUCOPON 220 UP can be obtained from Cognis Corporation, a business having offices in Ambler, Pa., and includes alkyl polyglycoside. The surfactant may be applied by any conventional means, such as dip and squeeze, spraying, printing, brush, foam, coating or the like. The surfactant may be applied to the entire laminate or may be selectively applied to particular sections of the laminate, such as the medial section along the longitudinal centerline of a diaper or other personal care product, to provide greater wettability of such sections. Exemplary surface treatment compositions and methods of applying surface treatment compositions are described in U.S. Pat. Nos. 5,057,361; 5,683,610 and 6,028,016 which also are hereby incorporated by reference herein.

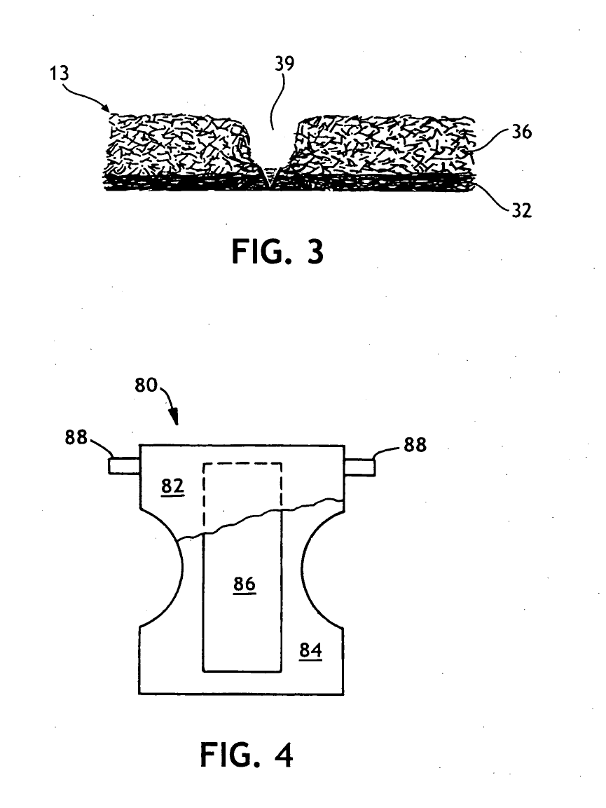

Fabrics of the present invention may be used in various personal care products, for example diapers. More specifically, the fabric of this invention may be used as a bodyside liner, core wrap or transfer layer in a diaper. A nonwoven fabric laminate of the present invention, for example the three-layer laminate 12 illustrated and described with reference to FIG. 2, a two-layer laminate 13 illustrated and described with reference to FIG. 3 or a laminate of additional layers, may be used in a wide variety of applications, not the least of which includes personal care absorbent articles such as diapers, training pants, incontinence devices and feminine hygiene products such as sanitary napkins. An exemplary article 80, in this case a diaper, is shown generally in FIG. 4 of the drawings. Other more complicated diaper constructions are known and are described and illustrated in greater detail in for example U.S. Pat. No. 5,520,673 to Yarbrough et al. and U.S. Pat. No. 6,217,890 to Paul et al., both of which are herby incorporated be reference herein. Referring to FIG. 4 of the present invention, most such personal care absorbent articles 80 include a liquid permeable top sheet or liner 82, a back sheet or outercover 84 and an absorbent core 86 disposed between and contained by the top sheet 82 and back sheet 84. Articles 80 such as diapers may also include some type of fastening means 88 such as adhesive fastening tapes or mechanical hook and loop type fasteners.

Specific examples of disposable diapers suitable for use in the present invention, and other components suitable for use therein, are disclosed in the following U.S. patents and U.S. patent applications: U.S. Pat. No. 4,798,603 issued Jan. 17, 1989, to Meyer et al.; U.S. Pat. No. 5,176,668 issued Jan. 5, 1993, to Bernardin; U.S. Pat. No. 5,176,672 issued Jan. 5, 1993, to Bruemmer et al.; U.S. Pat. No. 5,192,606 issued Mar. 9, 1993, to Proxmire et al.; U.S. Pat. No. 5,415,644 issued May 16, 1995, to Enloe; and U.S. Pat. No. 5,509,915 all of which are hereby incorporated herein by reference. Other suitable components include, for example, containment flaps and waist flaps. Specific examples of stretchable outercovers or backsheets that can be combined with liners, transfer layers and or core wraps of the present invention to produce a more stretchable diaper or other personal care article are described in U.S. Pat. No. 6,479,154 and U.S. patent application Ser. Nos. 10/703,761 and 10/918,553.

A nonwoven fabric laminate of the present invention by itself, or in other forms, such as a component in multilayer laminate including additional layers or some other composite structure, may be used to form various portions of the article including, but not limited to, the top sheet 82, as a wrap for the absorbent core 86 or a layer between the absorbent core 86 and the interior of the absorbent article 80, that is as a layer between the absorbent core 86 and a wearer of the article. In one example a laminate of the present invention is a wrap for the absorbent core 86 and/or the liner 82 portion of the diaper and can be formed completely from or include one of the laminates described herein to minimize the migration of particles from the absorbent core to the wearer's skin. If a laminate of the present invention is to be used as a top sheet 82, a core wrap material or as a layer between the absorbent core and a wearer, the laminate is desirably liquid permeable while retaining absorbent particles that may be contained in the absorbent core 86. Absorbent particles may have diameters as small as 0.001 inches; therefore, it would be desirable that the fine fiber layer of the laminate has holes or pores no larger than 0.001 inches in diameter. For example, a theoretically, perfect laid down grid of one micron polypropylene fibers would act as a barrier for 0.001 inch particles at a basis weight of 0.06 gsm. Thus, laminates of the present invention may include a fine fiber or meltblown layer having a basis weight of at least 0.06 grams per square meter (gsm). Laminates of the present invention with their fine fiber layers and resulting small pore size distribution can have superior particle retention and water vapor permeability properties.

As previously stated, laminates of the present invention can be used as a wrap for the absorbent core of an absorbent article or as a layer between the absorbent core and the interior of the absorbent article. For example, laminates of the present invention may be substituted as the core wrap material in a diaper such as the core wrap material described in U.S. Pat. No. 5,458,592 which is hereby incorporated be reference herein. As such, laminate materials of the present invention are particularly well-suited for containing absorbent cores which are made partially or completely from particulate matter such as superabsorbent particles. The laminate of the present invention is particularly useful for reducing the migration of superabsorbent particles in articles that contain superabsorbent particles. For example, the laminate is particularly useful in articles having an absorbent portion that has a high superabsorbent particle content such as greater than 50 weight percent. It should be understood, however, that the present invention is not restricted to use with superabsorbent particles but any particulate material such as odor absorbing and ion exchange resin particles and controlled release agents such as moisturizers, emollients and perfumes which require retention.

A “superabsorbent” or “superabsorbent material” refers to a water-swellable organic or inorganic material capable, under the most favorable conditions, of absorbing at least about 20 times its weight and, more desirably, at least about 30 times its weight in an aqueous solution containing 0.9 weight percent sodium chloride. Organic materials suitable for use as a superabsorbent material in conjunction with the present invention can include natural materials such as agar, pectin, guar gum, and so forth; as well as synthetic materials, such as synthetic hydrogel polymers. Such hydrogel polymers include, for example, alkali metal salts of polyacrylic acids, polyacrylamides, polyvinyl alcohol, ethylene maleic anhydride copolymers, polyvinyl ethers, methyl cellulose, carboxymethyl cellulose, hydroxypropylcellulose, polyvinylmorpholinone; and polymers and copolymers of vinyl sulfonic acid, polyacrylates, polyacrylamides, polyvinylpyrridine, and so forth. Other suitable polymers include hydrolyzed acrylonitrile grafted starch, acrylic acid grafted starch, and isobutylene maleic anhydride polymers and mixtures thereof. The hydrogel polymers are preferably lightly crosslinked to render the materials substantially water insoluble. Crosslinking may, for example, be accomplished by irradiation or by covalent, ionic, van der Waals, or hydrogen bonding. The superabsorbent materials may be in any form suitable for use in absorbent composites including particles, fibers, flakes, spheres, and so forth. Such superabsorbents are usually available in particle sizes ranging from about 20 to about 1000 microns. The absorbent core 86 can contain from 0 to 100 percent superabsorbent by weight based upon the total weight of the absorbent core. Typically an absorbent core 86 for a personal care absorbent product will include superabsorbent particles and, optionally, additional absorbent material such as absorbent fibers including, but not limited to, wood pulp fluff fibers, synthetic wood pulp fibers, synthetic fibers and combinations of the foregoing. Wood pulp fluff such as CR-1654 wood pulp available from Bowater Incorporated of Greenville, S.C. is an effective absorbent supplement. A common problem with wood pulp fluff, however, is its lack of integrity and its tendency to collapse when wet. As a result, it is often advantageous to add a stiffer reinforcing fiber into the absorbent core such as polyolefin meltblown fibers or shorter length staple fibers. Such combinations of fibers are sometimes referred to as “coform”. The manufacture of meltblown fibers and combinations of meltblown fibers with superabsorbents and/or wood pulp fibers are well known. Again, meltblown webs are made from fibers formed by extruding a molten thermoplastic material through a plurality of fine, usually circular dye capillaries as molten threads or filaments into a high-velocity heated air stream which attenuates the filaments of molten thermoplastic material to reduce their diameters. Shaped and/or multicomponent fibers may also be used. Thereafter, the meltblown fibers are carried by the high-velocity gas stream and are deposited on a collecting surface to form a web of randomly dispersed meltblown fibers. The meltblown process is well known and is described in various patents and publications, including NRL Report 4364, “Manufacture of Super-Fine Organic Fibers” by V. A. Wendt, E. L. Boone and C. D. Fluharty; NRL Report 5265, “An Improved Device For the Formation of Super-Fine Thermoplastic Fibers” by K. D. Lawrence, R. T. Lukas and J. A. Young; and U.S. Patent No. 3,849,241, issued Nov. 19, 1974 to Buntin et al. To form “coform” materials, additional components are mixed with the meltblown fibers as the fibers are deposited onto a forming surface. For example, superabsorbent particles and/or staple fibers and/or wood pulp fibers may be injected into the meltblown fiber stream so as to be entrapped and/or bonded to the meltblown fibers. See, for example, U.S. Pat. No. 4,100,324 to Anderson et al.; U.S. Pat. No. 4,587,154 to Hotchkiss et al., U.S. Pat. Nos. 4,604,313; 4,655,757 and 4,724,114 to McFarland et al. and U.K. Patent GB 2,151,272 to Minto et al., all of which are incorporated herein by reference in their entirety.

Laminates of the present invention that are intended to be used as a core wrap material or as a bodyside liner can be designed and formed to include a fibrous nonwoven web layer made from fine diameter thermoplastic fibers with particular pore sizes and air permeability. By thermoplastic fibers it is meant fibers which are formed from polymers such that the fibers can be bonded to themselves using heat or heat and pressure. While not being limited to the specific method of manufacture, meltblown fibrous nonwoven webs have been found to work particularly well. With respect to polymer selection, polyolefin fibers and especially polypropylene-based polymers have been found to work well. The general manufacture of such meltblown fibrous nonwoven webs is well known. See for example, the previously mentioned meltblown patents referred to above. The fibers may be hydrophilic or hydrophobic, though it is desirable that the resultant web, bodyside liner and core wrap be hydrophilic. As a result, the fibers may be formed from inherently wettable resins, such as polylactic acid, polyvinyl alcohol resins or polyesters, or can be treated to be hydrophilic as by the use of a surfactant treatment.

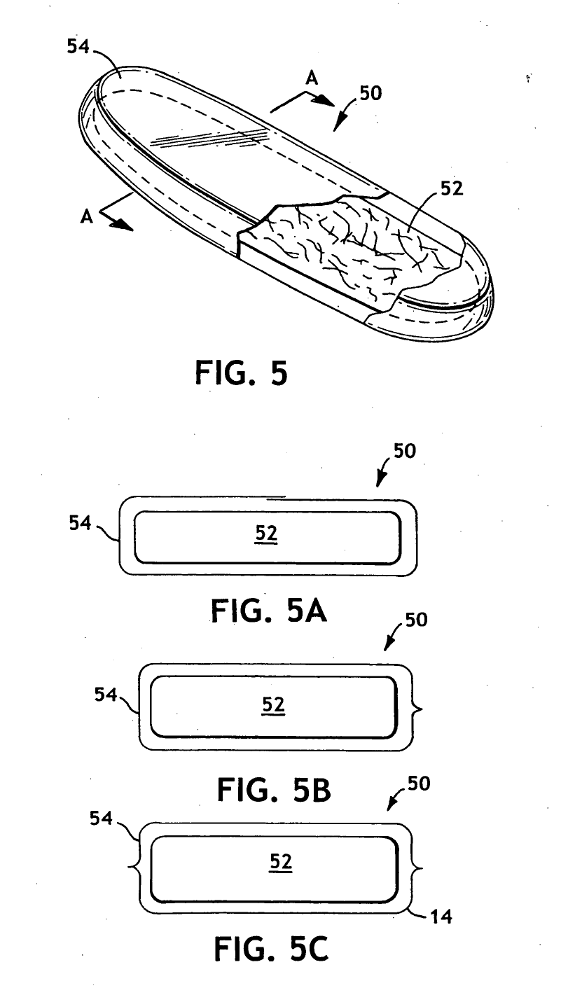

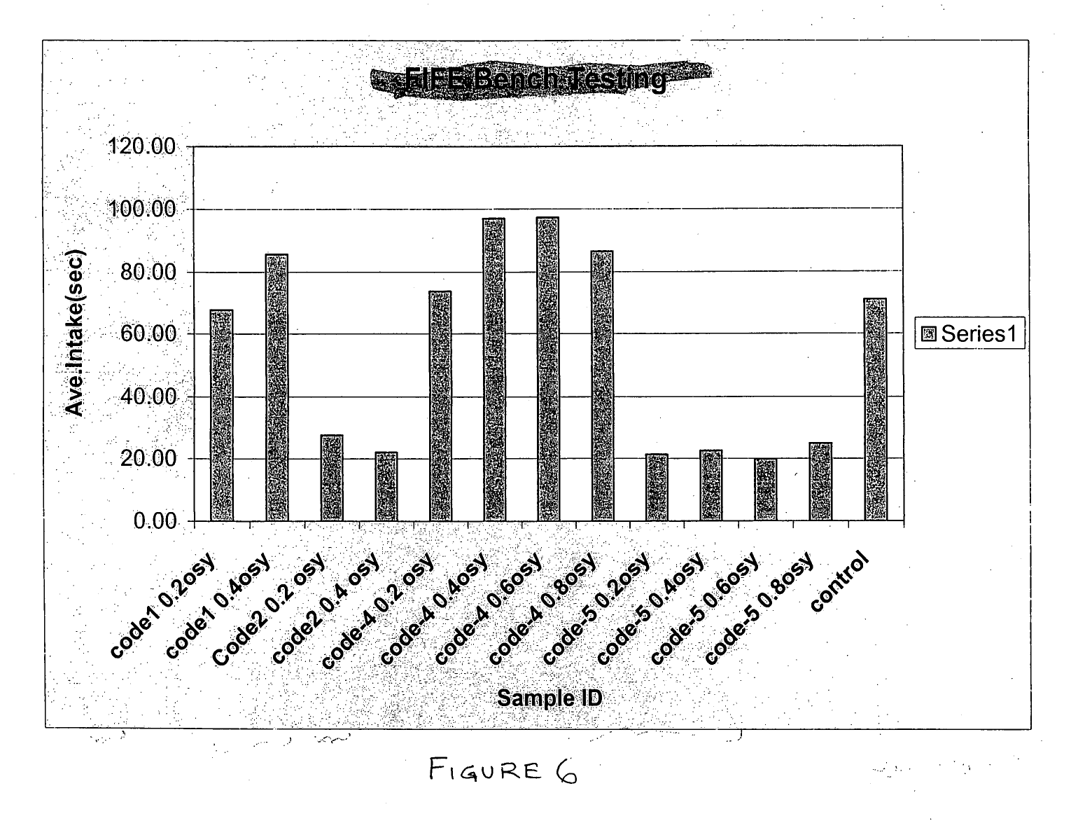

In order to function well as a core wrap, the meltblown web should have certain specific properties. A common problem with paper tissue wrap is that it has inadequate strength in the wet state. Typically a paper tissue wrap will have a wet to dry strength ratio in either the machine direction (MD) or cross-machine direction (CD) as measured by the test method outlined below of less than 0.5. In contrast, an absorbent core wrap of the present invention, illustrated as layer 54 in FIGS. 5, 5A, 5B and 5C, should have wet to dry strength ratios above 0.5 and sometimes 1.0 or higher. FIG. 5 illustrates a an absorbent core 52 that is wrapped or otherwise enveloped by a laminate of the present invention to form a wrapped absorbent core 50 that can be used as an absorbent portion of a diaper, for example, that does not release absorbent particles on the wearer.

The laminate wrap material 54 may be simply folded around the absorbent core 52 as illustrated in cross-sectional FIG. 5A, wrapped and bonded around one edge, as illustrated in FIG. 5B, two layers bonded around the absorbent core 52 as illustrated in FIG. 5C or unsealed. Alternatively, the laminate may be included as an additional layer in between the absorbent portion of an absorbent article and the skin contacting or body sideliner of the absorbent article. When using a two-layer nonwoven laminate such as that illustrated in FIG. 3 as the top sheet 82, it is may be advantageous to orient the fine fiber layer of laminate facing the absorbent core of the diaper to protect the fine fiber layer from damage and preserve the fine fiber layer's particle barrier properties. Although the present invention is illustrated by application to a diaper, laminates of the present invention can be used in other absorbent articles including, but not limited to, incontinence garments, pantiliners and so forth. Other potential uses for the multilayer nonwoven composite materials of the present invention may include, but are not limited to, surgical drapes, gowns, wipers, barrier materials, other garments and articles of clothing or portions thereof including such items as workwear and lab coats, and filtration materials such as water filters and so forth.

In one exemplary embodiment, the composite is heat activated to improve extensibility, processability and/or barrier properties of the composite. It is believe that the use of a low melting elastomer composition, such as a low-density metallocene-catalyzed polyethylene elastomer, in the meltblown layer in the composite allows the composite to be heat activated to soften the elastomeric fibers without significantly affecting the spunbonded layer(s) of the composite thus consolidating the composite web without degrading the meltblown layer. Generally, by low melting, we mean a polymer that melts and/or softens at a temperature significantly lower than the melting range of the polypropylene that is included in the composite fabric. This melting and/or softening temperature can easily be determined by reference to a DSC (differential scanning calorimeter) melting curve. The melting range of the selected metallocene-catalyzed elastomer can also be determined by the same DSC evaluation. Generally, it is desirable to select the metallocene-catalyzed elastomer and the polypropylene so as to provide a separation between the melting regions of the metallocene-catalyzed elastomer and the polypropylene. Suggested metallocene-catalyzed elastomers include, but are not limited to metallocene-catalyzed polyethylenes such as the Dow Affinity polymers. Other suggested elastomers include, but are not limited to, VERSIFY® Plastomers and Elastomers form Dow Chemical Company and Vistamaxx specialty elastomers from ExxonMobil Chemical Company of Houston, Tex. The inclusion of a low melting elastomeric meltblown layer and heat activation of the low melting elastomeric layer such a composite may be used to (1) improve the barrier properties of the composite by closing up the structure of the meltblown layer, (2) improve extensibility of the composite, (3) improve the softness of the composite, and/or (4) improve the bonding between the layers of the composite. It is believed that the use of low melting polymers improves processabilty because the inclusion of such low melting polymers allows the composite to be heat treated without stiffening or degrading the other layer(s) of the composite. The composite may be heat activated by exposing the composite nonwoven fabric to temperature sufficient to cause softening of the mPE component. In one embodiment, the composite nonwoven fabric is exposed to a higher temperature with for example by running the laminate through a hot air knife (HAK). An exemplary hot air knife (HAK) is described in U.S. Pat. No. 6,019,152 to Haynes et al.

Generally, it is desirable that the overall nonwoven composite fabric is extensible whether by necking, creping and/or including crimped fibers in the spunbonded layer(s) included in the composite fabric.

### EXAMPLES

A nonwoven composite in accordance with at least one embodiment of the present invention was made as follows. A lightweight layer consisted of meltblown (MB), elastic fibers having an average diameter of less than about 30 micrometers of basis weight of 0.2, 0.4, 0.6 and 0.8 osy (ounce per square yard), respectively, was deposited onto a first prenecked 0.45 osy extensible spunbonded layer to form 4 different composites (A, B, C and D) having different weight MB layers. The first spunbond layer consisted essentially of 100 weight percent of EXXON PD-3445 polypropylene and extruded through 0.6 mm holes at a rate of 0.7 grams/hole/minute (ghm) to form a first layer of spunbond fibers. The spunbonded layer was prenecked by about 60 percent. The meltblown layer was formed from a thermoplastic, elastic or elastomeric polymer composition consisting of KRATON® 2760 elastomer and the weight percent of surfactant(s) specified in the each specific example below. A surfactant or a mixture of surfactants was included in the KRATON®) 2760 elastomer by compounding the surfactant(s) in the KRATON® 2760 elastomer resin prior to the elastomer being extruded into the fine fibers of the meltblown layer. Three different surfactant compositions were used: (1) AHCOVEL (2) MASIL SF-19 and (3) a 3:1 weight mixture of AHCOVEL and MASIL SF-19. The components of the thermoplastic, elastic/elastomeric composition were compounded using a concentrated masterbatch method as is well known in the art. The compounded pellets were damp coming out of the extruder and were dried prior to being used to form the meltblown nonwoven layer on the first spunbond layer. In some of the examples the first spunbond layer was prenecked and/or precreped prior to the meltblown layer being deposited on the first spunbond layer. A second spunbonded layer similar to the first spunbond layer was deposited onto the meltblown layer of fibers. The three layers were passed through a pressurized nip to enhance interlayer adhesion between the exterior spubonded layers and the interior meltblown layer forming a spunbonded/meltblown/spunbonded (SMS) composite.

### Example 1A and 1B

### 0.2 and 0.4 Osy, Respectively

Necked spunbond layer(s) and no surfactant in KRATON® meltblown layer. SB layers treated with 1 percent AHCOVEL.

### Example 2A and 2B

### 0.2 and 0.4 Osy, Respectively

Necked and creped spunbond layer(s) treated with 1% AHCOVEL and no surfactant in KRATON® meltblown layer.

### Example 3B

### 0.4 Osy

Necked spunbond layer(s) and no surfactant in KRATON® meltblown layer. The entire nonwoven composite of Example 3B was measured for pore size distribution characterization to predict superabsorbent particle (SAP) strikethrough. Example 3B (having a 0.4 osy MB elastomeric layer) was measured for pore size and percent open area using the test method described above. The MB layer of Example 3B was measured as having an equivalent-circular diameter (ECD) of 40.52 μm and a standard deviation of 23.40. The convoluted pore width CPW was measured at 25.27 μm. The percent open area was measured at 11.63%. In addition, the area-weight CPW was 46.18 μm. In contrast, a standard SMS laminate with a 0.4-osy polyolefin MB layer made under similar conditions had an ECD of 47.26 μm with a standard deviation of 31.43, a CPW of 25.75 μm, a percent open area of 16.07 and area-weight CPW of 49.43 μm. A standard SB liner that consisted essentially of a 0.4-osy polypropylene MB layer made under similar conditions had an ECD of 46.21 μm with a standard deviation of 28.55, a CPW of 27.32 μm, a percent open area of 22.66 and area-weight CPW of 52.92 μm. Thus, a stretchable liner (Example 3B) can be made that have porosity properties equivalent to standard SMS and SB liners but that will also be stretchable in both the MD and CD directions.

### Example 4A 4B, 4C and 4D

### 0.2, 0.4, 0.6 and 0.8 Osy, Respectively

Necked spunbond layer(s) treated with 1 weight percent AHCOVEL surfactant in KRATON® meltblown layer.

### Example 5A 5B, 5C and 5D

### 0.2, 0.4, 0.6 and 0.8 Osy, Respectively

Necked and creped spunbond layer(s) treated with 1 weight percent AHCOVEL surfactant in KRATON® meltblown layer.

### Example 6

Necked spunbond layer(s) and 3 weight percent AHCOVEL surfactant in KRATON® meltblown layer.

### Example 7

Necked and creped spunbond layer(s) and 3 weight percent AHCOVEL surfactant in KRATON® meltblown layer.

### Example 8

Necked spunbond layer(s) treated with 1 weight percent Dow Corning193 Surfactant in KRATON® meltblown layer.

### Example 9

Necked and creped spunbond layer(s) treated with 3 weight percent AHCOVEL surfactant in KRATON® meltblown layer.

### Example 10

Necked spunbond layer(s) and 1 weight percent of a 3:1 weight mixture of AHCOVEL and Dow Corning193 Surfactant in KRATON® meltblown layer.

### Example 11

Necked and creped spunbond layer(s) and 1 weight percent of a 3:1: weight mixture of AHCOVEL and Dow Corning 193 Surfactant in KRATON® meltblown layer.

### Example 12

Necked spunbond layer(s) and 3 weight percent of a 3:1 weight mixture of AHCOVEL and Dow Corning 193 Surfactant in KRATON® meltblown layer.

### Example 13

Necked spunbond layer(s) and 1 weight percent of a 3:1 weight mixture of AHCOVEL and MASIL SF-19 surfactants in KRATON® meltblown layer.

### Control Example A

Control Example A was a conventional diaper liner that consisted of a standard polypropylene liner externally treated with 1% AHCOVEL.

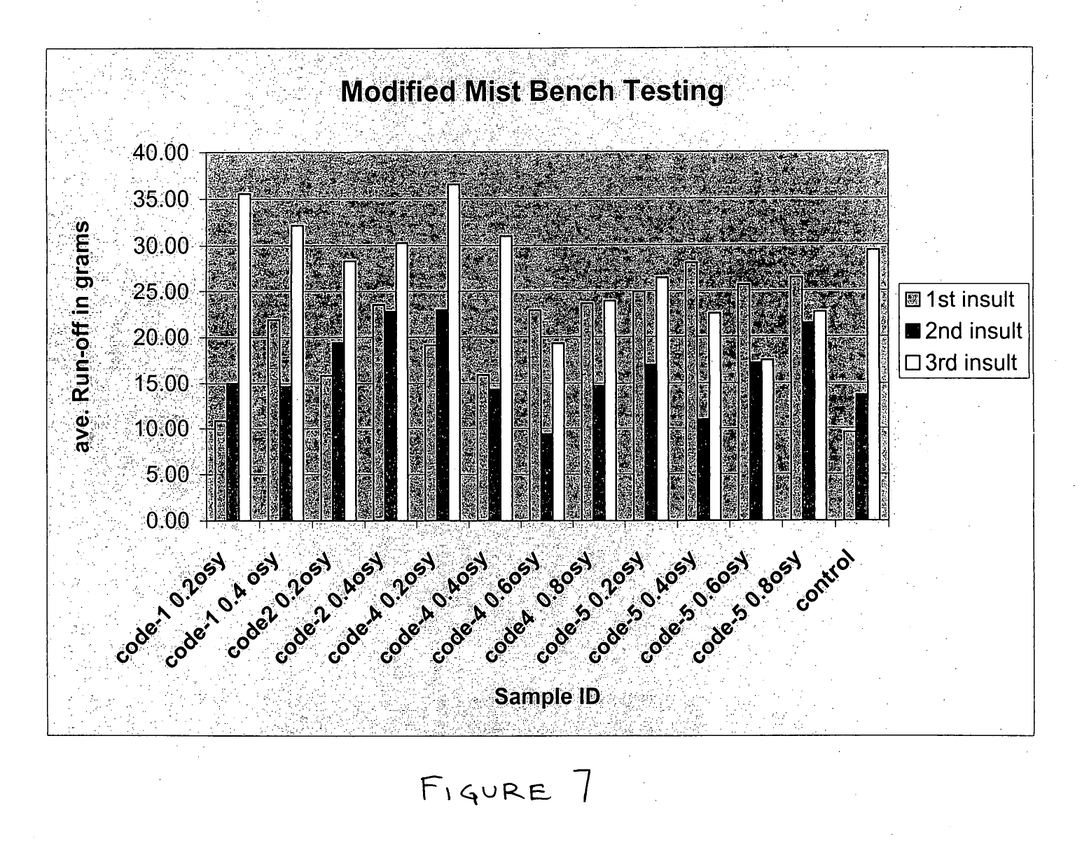

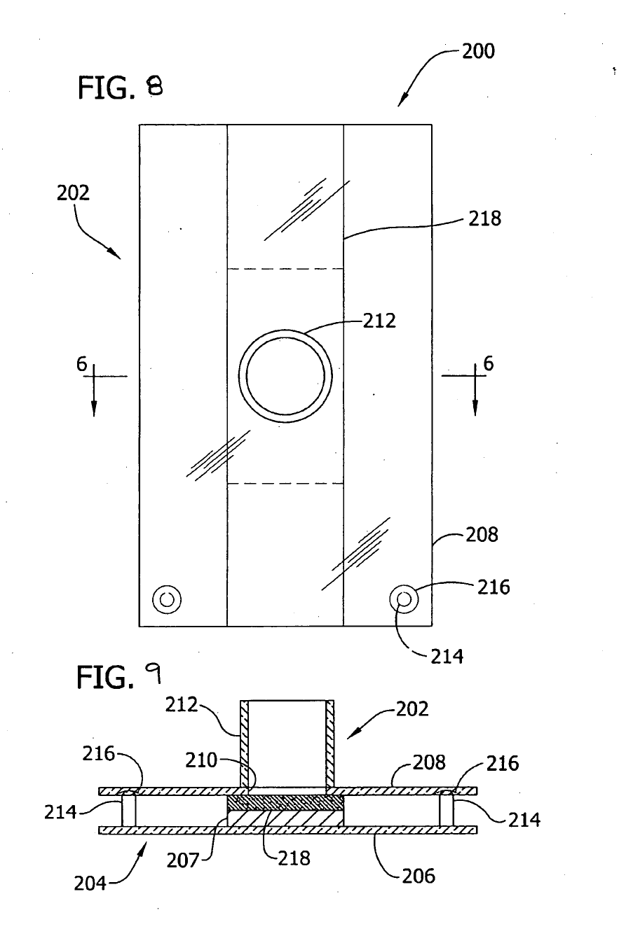

Several examples were tested for FIFE intake, modified mist run-off and air permeability using the test procedures described above. The results of the FIFE test are presented in FIG. 6 and the results of the modified mist testing are presented in FIG. 7.

We claim:

1. A personal care product comprising:
a. a liquid impervious backing sheet,
b. a nonwoven fabric composite that comprises at least one layer of fine fibers and at least one layer of spunbonded fibers wherein the at least one layer of spunbonded layers is extensible and the at least one layer of fine fibers comprises fibers that comprise a blend of an elastomeric polymer and an internal wetting agent, and
c. an absorbent material disposed between the liquid impervious backing sheet and the nonwoven fabric composite.

2. The personal care product of claim 1 wherein the layer of fine fibers consists essentially of meltblown elastomeric fibers.

3. The personal care product of claim 1 wherein nonwoven fabric composite further comprises a second layer of spunbonded fibers wherein the second layer of spunbonded layers is extensible.

4. The personal care product of claim 3 wherein the layer of fine fibers consists essentially of a layer of meltblown elastomeric fibers and the nonwoven fabric composite consists essentially of the layer of fine fibers disposed between two spunbonded layers.

5. The personal care product of claim 1 wherein the nonwoven fabric composite is a liquid pervious bodyside liner or a layer between the absorbent material and a liquid pervious bodyside liner.

6. The personal care product of claim 1 wherein the nonwoven fabric composite is a liquid pervious bodyside liner.

7. The personal care product of claim 1 wherein the personal care product further comprises a liquid pervious bodyside liner and the nonwoven fabric composite is a layer between the absorbent material and the liquid pervious body side liner.

8. The personal care product of claim 1 wherein the nonwoven fabric composite envelops the absorbent material.

9. The personal care product of claim 1 wherein the absorbent material comprises particles selected from the groups consisting of superabsorbent particles, synthetic polymer particles, carbon particles and combinations thereof.

10. The personal care product of claim 1 wherein the layer of fine fibers consists essentially of a layer of fine fibers selected from the group of metallocene polyethylene fibers and styrenic block copolymer fibers and mixtures thereof.

11. The personal care product of claim 1 wherein the layer of fine fibers consists essentially of a layer of fine fibers selected from the group styrene/ethylene-propylene/styrene/ethylene-propylene tetrablock copolymer fibers and styrene/ethylene-butylene/styrene/ethylene-butylene tetrablock copolymer fibers and mixtures thereof.

12. The personal care product of claim 1 wherein the internal wetting agent is selected from the group consisting of modified castor oils, hydrogenated ethoxylated castor oils, sorbitan monooleate, alkyl polyglycosides, ethoxylated siloxanes and mixtures thereof.

13. The personal care product of claim 1 wherein the absorbent material comprises particles of a superabsorbent material having an average diameter that is greater than the average diameter of the pores in the layer of meltblown layer.

14. The personal care product of claim 1 wherein the nonwoven fabric composite is necked, creped or crimped.

15. The personal care product of claim 1, wherein the personal care product is a diaper, a training pant, an absorbent underpant, an adult incontinence products or a feminine hygiene product.

16. A nonwoven fabric composite consisting essentially of:
a. an extensible first layer of spunbonded fibers,
b. an extensible second layer of spunbonded fibers,
c. a layer of meltblown fibers disposed between the first layer of spunbonded fibers and the second layer of spunbonded fibers, wherein the layer of meltblown fibers consist essentially of elastomeric fibers include an internal wetting agent or are treated with a wetting agent.

17. The nonwoven fabric composite of claim 16 wherein the layer of fine fibers consists essentially of a layer of fine fibers selected from the group of metallocene polyethylene fibers and styrenic block copolymer fibers.

18. The nonwoven fabric composite of claim 16 wherein the layer of fine fibers consists essentially of a layer of fine fibers selected from the group of styrene/ethylene-propylene/styrene/ethylene-propylene tetrablock copolymer fibers and styrene/ethylene-butylene/styrene/ethylene-butylene tetrablock copolymer fibers.

19. The nonwoven fabric composite of claim 16 wherein the first spunbonded layer, the meltblown layer and the second spunbonded layer are intermittently bonded to form the nonwoven fabric composite.

20. The nonwoven fabric composite of claim 16 wherein the first spunbonded layer, the meltblown layer and the second spunbonded layer are densified with a hot air knife.

21. The nonwoven fabric composite of claim 16 wherein the internal wetting agent is selected from the group consisting of modified castor oils, hydrogenated ethoxylated castor oils, sorbitan monooleate, alkyl polyglycosides, ethoxylated siloxane and mixtures thereof.

22. The nonwoven fabric composite of claim 16 wherein the nonwoven fabric composite is necked, creped or crimped.
